(19) United States
(12) Patent Application Publication
(10) Pub. No.: US 2022/0090165 A1
(43) Pub. Date: Mar. 24, 2022

(54) METHOD OF COLLECTING NUCLEIC ACID AND KIT FOR COLLECTION OF NUCLEIC ACID

(71) Applicant: Toray Industries, Inc., Tokyo (JP)

(72) Inventors: Shota Sekiguchi, Kamakura-shi, Kanagawa (JP); Takahiro Motoshiromizu, Kamakura-shi, Kanagawa (JP); Masateru Ito, Kamakura-shi, Kanagawa (JP)

(21) Appl. No.: 17/287,194

(22) PCT Filed: Oct. 21, 2019
(86) PCT No.: PCT/JP2019/041410

§ 371 Date: Apr. 21, 2021

(30) Foreign Application Priority Data
Oct. 23, 2018 (JP) 2018-199267

(51) Int. Cl. C12Q 1/6806; G01N 30/14; G01N 30/88

(52) U.S. Cl. CPC C12Q 1/6806; G01N 2030/8827; G01N 30/88; G01N 30/14

(57) ABSTRACT

A method of collecting a nucleic acid from a sample containing a nucleic acid using a support of aluminum oxide having a surface where a water-soluble neutral polymer is adsorbed, the method including steps a to c: step a: a step of bringing the support into contact with the sample containing a nucleic acid to adsorb the nucleic acid on the support; step b: a step of bringing the support on which the nucleic acid is adsorbed into contact with a solution A containing 1 mM or more and 40 mM or less of a chelating agent; and step c: after the step b, a step of bringing the support on which the nucleic acid is adsorbed into contact with a solution B containing 50 mM or more of a chelating agent to elute the nucleic acid.

### SEQUENCE LISTING

The instant application contains a Sequence Listing which has been submitted electronically in ASCII format and is hereby incorporated by reference in its entirety. Said ASCII copy, created on Oct. 4, 2021, is named SIP-21-1114_373207-000066_SL.txt and is 846 bytes in size.

### TECHNICAL FIELD

This disclosure relates to a method of collecting a nucleic acid at a high yield from a sample containing a nucleic acid using a support of aluminum oxide having a surface where a water-soluble neutral polymer is adsorbed, and a kit for collection of a nucleic acid.

### BACKGROUND

Development of experimental techniques using nucleic acids has enabled a novel gene search and analysis of the gene. In clinical sites, a screening test and a clinical test using gene analysis have been performed. The tests are used to identify a disease such as cancer, or to identify infection of a pathogen. In such tests using gene analysis, a gene collected from a body fluid sample such as blood or urine is used. Therefore, the tests are expected to be a minimally invasive test.

As a target of gene analysis in such a body fluid, not only long-chain nucleic acids such as a genome, but also short-chain nucleic acids of 1,000 bases or less have attracted attention. miRNAs that have been discovered in recent years are single-stranded RNAs of 18 bases or more and 25 bases or less, and are biosynthesized from pre-miRNAs of 60 bases or more and 90 bases or less. These nucleic acids are considered to be involved in the disease since they have a function of controlling synthesis of a protein and gene expression. They have particularly attracted attention as a target of gene analysis capable of early detection of cancer. Cell-free DNAs that have attracted attention in recent years are double-stranded DNAs having a length about one to four times 166 bases that correspond to one unit of histone, and are produced through extinction and decomposition of cells. Among the cell-free DNAs, in particular, cell-free DNAs derived from cancer cells are called ctDNAs. The ctDNAs have a cancer-specific genetic mutation. Therefore, the ctDNAs have attracted attention as a target for judgement of the presence or absence of effect on a therapeutic agent, or for determination of the presence or absence of cancer.

International Publication WO 2016/152763 discloses a method of collecting a nucleic acid from a sample containing a nucleic acid using a support of aluminum oxide on which a water-soluble neutral polymer is adsorbed. Specifically, as illustrated in FIG. 2 described below, a nucleic acid is adsorbed on a support, an eluent is added to the support on which the nucleic acid is adsorbed, to elute the nucleic acid, and as a result, the nucleic acid is collected.

In recent years, a variety of nucleic acids are to be analyzed, and a trace amount of nucleic acid present in a body fluid may also be analyzed. Therefore, a method of collecting a nucleic acid at a high yield as compared with a conventional method is required.

The method of collecting a nucleic acid described in WO '763 has attracted attention in terms of a method capable of collecting a nucleic acid at a relatively high yield. However, a method of collecting a nucleic acid at a further high yield is required.

It could therefore be helpful to provide a method of collecting a nucleic acid from a sample containing a nucleic acid at a high yield, namely a method that particularly enables collection of a trace amount of nucleic acid present in a body fluid at a high yield, and a kit for collection of a nucleic acid.

### SUMMARY

We investigated methods capable of collecting a nucleic acid at a higher yield based on the method of collecting a nucleic acid from a sample containing a nucleic acid disclosed in WO '763. We found that when a step of bringing a support on which a nucleic acid is adsorbed into contact with a solution containing 1 mM or more and 40 mM or less of a chelating agent is added as a step prior to addition of an eluent to the support on which the nucleic acid is adsorbed, the amount of nucleic acid collected is further increased.

We thus provide:

(1) A method of collecting a nucleic acid from a sample containing a nucleic acid using a support of aluminum oxide having a surface where a water-soluble neutral polymer is adsorbed, the method including following steps a to c:

step a: a step of bringing the support into contact with the sample containing a nucleic acid to adsorb the nucleic acid on the support;

step b: a step of bringing the support on which the nucleic acid is adsorbed into contact with a solution A containing 1 mM or more and 40 mM or less of a chelating agent; and

step c: after the step b, a step of bringing the support on which the nucleic acid is adsorbed into contact with a solution B containing 50 mM or more of a chelating agent to elute the nucleic acid.

(2) The method of collecting a nucleic acid according to (1), wherein the chelating agent is a carboxylic acid-based chelating agent, a phosphoric acid-based chelating agent, or a phosphonic acid-based chelating agent.
(3) The method of collecting a nucleic acid according to (2), wherein the carboxylic acid-based chelating agent is citric acid, nitrilotriacetic acid, ethylenediaminetetraacetic acid, glycol ether diaminetetraacetic acid, and/or a salt thereof.
(4) The method of collecting a nucleic acid according to (2), wherein the phosphoric acid-based chelating agent is phosphoric acid, polyphosphoric acid, metaphosphoric acid, and/or a salt thereof.
(5) The method of collecting a nucleic acid according to (2), wherein the phosphonic acid-based chelating agent is 1-hydroxyethane-1,1-diphosphonic acid, glycine-N,N-bis(methylene-phosphonic acid), nitrilotris(methylenephosphonic acid), 2-phosphonobutane-1,2,4-tricarboxylic acid, ethylenediamine tetramethylenephosphonic acid, and/or a salt thereof.
(6) The method of collecting a nucleic acid according to any of (1) to (5), wherein the support is housed in a column for use.
(7) The method of collecting a nucleic acid according to any of (1) to (6), wherein the water-soluble neutral polymer is a polymer having a zeta potential of −10 mV or more and +10 mV or less in a solution with a pH of 7.
(8) The method of collecting a nucleic acid according to any of (1) to (7), wherein the water-soluble neutral polymer is polyethylene glycol, polyvinyl alcohol, polyvinylpyrrolidone, poly(2-ethyl-2-oxazoline), or (hydroxypropyl)methylcellulose.
(9) A kit for collection of a nucleic acid, the kit including a support of aluminum oxide having a surface where a water-soluble neutral polymer is adsorbed, a solution A containing 1 mM or more and 40 mM or less of a chelating agent, and a solution B containing 50 mM or more of a chelating agent.

A nucleic acid can be collected at a high yield compared to conventional methods. Therefore, it is expected to enable collection of a trace amount of nucleic acid present in a body fluid and collection of a novel nucleic acid.

### BRIEF DESCRIPTION OF THE DRAWINGS

FIG. 1 is a flowchart illustrating an outline of each step in a method of collecting a nucleic acid according to one example.

FIG. 2 is a flowchart illustrating an example of a method of collecting a nucleic acid described in WO '763.

### DETAILED DESCRIPTION

We provide a method of collecting a nucleic acid from a sample containing a nucleic acid using a support of aluminum oxide having a surface where a water-soluble neutral polymer is adsorbed, the method including the following steps a to c:

step a: a step of bringing the sample containing a nucleic acid into contact with the support to adsorb the nucleic acid on the support;

step b: a step of bringing a solution A (first solution) containing 1 mM or more and 40 mM or less of a chelating agent into contact with the support on which the nucleic acid is adsorbed; and

step c: after the step b, a step of bringing a solution B (second solution) containing 50 mM or more of a chelating agent into contact with the support on which the nucleic acid is adsorbed, to elute the nucleic acid.

Between the steps a and b and between the steps b and c, a washing step of washing a product after a treatment is performed.

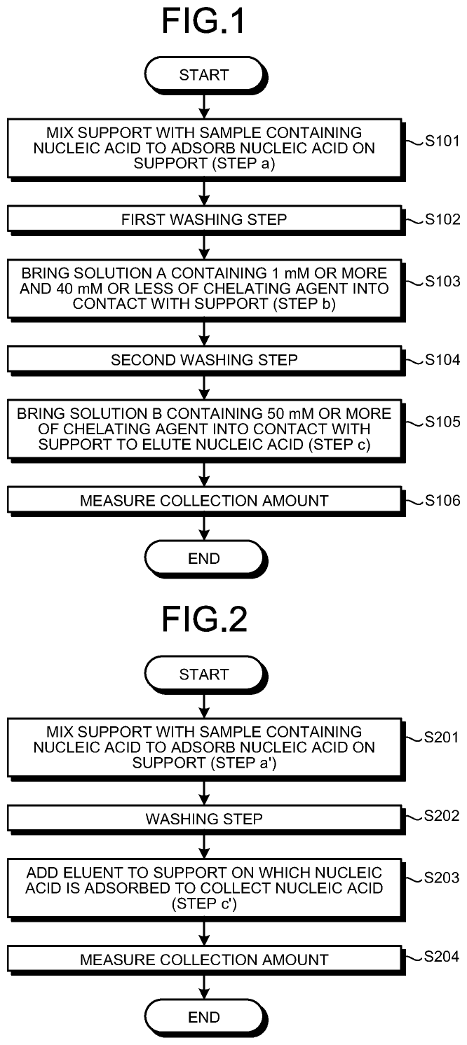

Specific treatment processes in the method of collecting a nucleic acid will be described with reference to FIG. 1. FIG. 1 is a flowchart illustrating an outline of each step in a method of collecting a nucleic acid according to one example.

The sample containing a nucleic acid is brought into contact with the support, to adsorb the nucleic acid on the support (the step a: Step S101).

After the sample is brought into contact with the support, a washing treatment is performed to remove a substance derived from the sample other than the nucleic acid, and the like from the support (a first washing step: Step S102).

After the first washing step, the solution A containing 1 mM or more and 40 mM or less of a chelating agent is brought into contact with the support on which the nucleic acid is adsorbed (the step b: Step S103).

After the sample is brought into contact with the support, the washing treatment is performed to remove the chelating agent and the like after the contact treatment (a second washing step: Step S104).

After the second washing step, the solution B (second solution) containing 50 mM or more of a chelating agent is brought into contact with the support on which the nucleic acid is adsorbed, to elute the nucleic acid (the step c: Step S105).

Subsequently, the collection amount of the nucleic acid adsorbed on the support is measured (Step S106). At Step S106, the amount of nucleic acid eluted is calculated as the collection amount.

The support of aluminum oxide having a surface where a water-soluble neutral polymer is adsorbed is sometimes referred to as “our support.”

On the other hand, the method of collecting a nucleic acid described in WO '763 is a method including a step a′ and a step c′ respectively corresponding to the step a and the step c as basic steps. The steps a′ and c′ are as follows:

Step a′: a step of mixing a support of aluminum oxide having a surface where a water-soluble neutral polymer is adsorbed with a solution containing a nucleic acid, to adsorb the nucleic acid on the support.

Step c′: a step of adding an eluent to the support on which the nucleic acid is adsorbed, to collect the nucleic acid.

Between the steps a′ and c′, a washing step of washing a compound after a treatment is performed.

Specific treatment processes in the conventional method of collecting a nucleic acid will be described with reference to FIG. 2. FIG. 2 is a flowchart illustrating an example of the method of collecting a nucleic acid described in WO '763.

The support of aluminum oxide having a surface where a water-soluble neutral polymer is adsorbed is first mixed with the solution containing a nucleic acid, to adsorb the nucleic acid on the support (the step a′: Step S201).

After the support is mixed with the solution, the washing treatment is performed, to remove a substance derived from the sample other than the nucleic acid, and the like from the support (a washing step: Step S202).

After the washing step, the eluent is added to the support on which the nucleic acid is adsorbed, to collect the nucleic acid (the step c′: Step S203).

Subsequently, the collection amount of the nucleic acid adsorbed on the support is measured (Step S204). At Step S204, the amount of nucleic acid eluted is calculated in the same manner as that at Step S106, for example.

We specified that the solution B containing 50 mM or more of a chelating agent is added as an eluent that causes elution of the nucleic acid at the step c, as illustrated in FIG. 1. Furthermore, we found that when as a previous step of the step c, the step b of bringing the solution A containing 1 mM or more and 40 mM or less of a chelating agent into contact with the support on which the nucleic acid is adsorbed, and removing the solution A is added, the nucleic acid can be collected at a high yield. Hereinafter, our method will be described for each step.

The step a is a step of bringing the sample containing a nucleic acid into contact with our support, to adsorb the nucleic acid on our support.

A method of bringing the sample containing a nucleic acid into contact with our support is not particularly limited, and examples thereof include a method in which our support is housed in a column, and the sample containing a nucleic acid is passed through the column, a mixing method with a pipetter, a mixer, a vortex, or the like, and a mixing method by inversion. Among these methods, the method in which our support is housed in a column, and the sample containing a nucleic acid is passed through the column is preferred.

The shape of the column that houses our support is not particularly limited. A column that houses our support on an ultrafiltration membrane or a mesh having a smaller pore diameter than the particle diameter of our support can be used. For example, our support is housed in a centrifugal filtration kit such as “Ultrafree” (registered trademark) manufactured by Merck Ltd., or “Nanosep” (registered trademark) manufactured by Pall Corporation, and the centrifugal filtration kit can also be used as a column that houses our support.

Examples of a method of passing a liquid through the column include a method in which the pressure in the column is changed in a positive pressure by using a pump, a centrifugal separator, or the like, to pass a liquid through the column, a method of passing a liquid through the column by gravity without a pump or the like, and a method in which the pressure on a discharge side of the column is changed in a negative pressure by using a suction pump or the like, to pass a liquid through the column. Any of the methods may be utilized. The time required for passing a liquid through the column is preferably 90 minutes or less.

After an operation of the step a, the following washing treatment is performed. This is because when the sample containing a nucleic acid is a biological sample, it is possible that a substance derived from the sample other than a target nucleic acid be adsorbed on a surface of our support after the step a. When the substance derived from the sample other than the nucleic acid is cleaned or decomposed, the nucleic acid can be collected at a higher purity. Specifically, various treatments such as washing with water to remove a non-specifically adsorbed compound, washing with a surfactant to remove a non-specifically adsorbed protein, washing with a solution containing a nonionic surfactant to remove an ion and a low-molecular-weight compound, washing with an organic solvent to remove a non-specifically adsorbed hydrophobic compound, addition of a protease to decompose a non-specifically adsorbed protein, addition of an RNase to isolate only a DNA, and addition of a DNase to isolate only an RNA can be performed. In FIG. 1, this washing treatment is represented as the first washing step. This first washing step may be performed, if necessary. When the first washing step is unnecessary, Step S103 is performed after Step S101.

The step b is a step of bringing the solution A containing 1 mM or more and 40 mM or less of a chelating agent into contact with the support on which the nucleic acid is adsorbed at the step a.

The chelating agent is a substance for which a substance that has a ligand with a plurality of configuration coordinates and binds to a metal ion to form a complex can be used.

The chelating agent is classified depending on an ionic functional group of the chelating agent. Specifically, the chelating agent is classified into a carboxylic acid-based chelating agent such as an aminocarboxylic acid-based, hydroxycarboxylic acid-based, hydroxyaminocarboxylic acid-based, or ethercarboxylic acid-based chelating agent, a phosphoric acid-based chelating agent, an ether-based chelating agent, an amine-based chelating agent and the like. Among these chelating agents, a carboxylic acid-based or phosphoric acid-based chelating agent is preferred.

Specific examples of the aminocarboxylic acid-based chelating agent include nitrilotriacetic acid (NTA), ethylenediaminetetraacetic acid (EDTA), glycol ether diaminetetraacetic acid (EGTA), diethylenetriaminopentaacetic acid (DTPA), and/or salts thereof. Specific examples of the hydroxycarboxylic acid-based chelating agent include oxalic acid, citric acid, gluconic acid, tartaric acid, and/or salts thereof. Specific examples of the hydroxyaminocarboxylic acid-based chelating agent include dihydroxyethylglycine (DEG), N-(2-hydroxyethyl)iminodiacetic acid (HEIDA), hydroxyethylethylenediaminetetraacetic acid (HEDTA), and/or salts thereof. Specific examples of the ethercarboxylic acid-based chelating agent include carboxymethyltartronic acid (CMT), carboxymethyloxysuccinic acid (CMOS), and/or salts thereof. In particular, citric acid, nitrilotriacetic acid, ethylenediaminetetraacetic acid, glycol ether diaminetetraacetic acid, and/or salts thereof are preferred.

Specific examples of the phosphoric acid-based chelating agent include phosphoric acid, polyphosphoric acid, metaphosphoric acid, phytic acid, and/or salts thereof. In particular, phosphoric acid, polyphosphoric acid, metaphosphoric acid, and/or salts thereof are preferred. Polyphosphoric acid is a linear condensed phosphoric acid represented by a general formula of (PₙO₃ₙ₊₁)(n≥2). In particular, the polyphosphoric acid, where n is 2, is also called pyrophosphoric acid, and the polyphosphoric acid, where n is 3, is also called triphosphoric acid. When n is larger, the polyphosphoric acid has an anion (PₙO₃ₙ)ⁿ⁻ in which a long structure of “—O—P—O—P—O— . . . ” is helically connected, and is called metaphosphoric acid. The metaphosphoric acid may have a cyclic structure. Polyphosphoric acid, metaphosphoric acid, and/or a salt thereof that have any structure can be preferably used as a phosphoric acid-based chelating agent, and a mixture thereof can also be preferably used.

Specific examples of a phosphonic acid-based chelating agent include 1-hydroxyethane-1,1-diphosphonic acid (HEDP), glycine-N,N-bis(methylenephosphonic acid) (GMP), nitrilotris(methylenephosphonic acid) (NTMP), 2-phosphonobutane-1,2,4-tricarboxylic acid (PBTC), ethylenediamine tetramethylenephosphonic acid (EDTMP), and/or salts thereof.

Among the chelating agents, one kind of chelating agent may be used, or a mixture of two or more kinds of chelating agents may be used for the solution A. When the mixture of two or more kinds of chelating agents is used, it is preferable that a mixture of phosphoric acid and polyphosphoric acid and/or salts thereof, phosphoric acid and metaphosphoric acid and/or salts thereof, or phosphoric acid and phytic acid and/or salts thereof be used.

As the solution A, a solution in which the chelating agent is dissolved to a concentration of 1 mM or more and 40 mM or less is used. In a carboxylic acid-based chelating agent, the concentration is more preferably 5 mM or more and 25 mM or less. In a phosphoric acid-based chelating agent, the concentration is more preferably 1 mM or more and 10 mM or less. For a solvent, water, a neutral to alkali aqueous solution, or a buffer solution can be used. The solution containing a chelating agent can also be prepared by neutralizing a free form of the carboxylic acid-based or phosphoric acid-based chelating agent to form a salt. For example, a solution containing citric acid as a chelating agent can be prepared by dissolving citric acid in water, an aqueous sodium hydroxide solution, a HEPES buffer solution, or the like. The solution containing citric acid can also be prepared by dissolving a sodium salt of citric acid in water, an aqueous hydrochloric acid solution, a HEPES buffer solution, or the like. The solution containing citric acid can also be prepared by mixing an aqueous citric acid solution with an aqueous solution of sodium citrate.

The pH of the solution A is preferably 4 or more and 9 or less, and more preferably 5 or more and 8 or less.

As the solution A, a solution prepared at the time of use may be used, or a solution prepared in advance may be used.

For the step of bringing the solution A into contact with the support on which the nucleic acid is adsorbed, and removing the solution A at the step b, a method of bringing the solution A into contact with the support can be performed in the same manner as that at the step a. When at the step a, the column that houses our support is used to adsorb the nucleic acid on our support, by passing the solution A through the column that houses the support on which the nucleic acid is adsorbed, bringing the solution A into contact with the support and removing the solution A from the support are performed by one operation, which is preferable. When the column is used, the liquid passing time is preferably 10 minutes or less. When the solution A is brought into contact with the support on which the nucleic acid is adsorbed by a mixing method with a pipetter, a mixer, a vortex, or the like, or a mixing method by inversion, a method in which a mixture obtained by mixing is centrifuged, the support on which the nucleic acid is adsorbed is precipitated, and the supernatant is removed can be used. Since the specific gravity of the support on which the nucleic acid is adsorbed is higher than that of water, the support can be easily precipitated by centrifugation. The centrifugation may be performed at 6,000 G for 1 minute, and more preferably at 10,000 G for 1 minute.

After an operation at the step b, the washing treatment is performed in the same manner as that at the aforementioned washing step 1. This is because when the chelating agent used at the step b remains in a system, the concentration of the chelating agent in the solution after collection of the nucleic acid differs from the addition concentration, which may affect a subsequent measurement system. In FIG. 1, this washing treatment is represented as the second washing step. This second washing step may be performed, if necessary. When the second washing step is unnecessary, Step S105 is performed after Step S103 described above.

The step c is a step of bringing the solution B containing 50 mM or more of a chelating agent into contact with our support on which the nucleic acid is adsorbed after the step b. The solution B is an eluent for eluting the nucleic acid from our support on which the nucleic acid is adsorbed.

The solution B can be prepared in the same manner as the method of preparing the solution A except that the concentration of the chelating agent is adjusted to 50 mM or more.

The pH of the solution B is preferably 4 or more and 9 or less, and more preferably 5 or more and 8 or less.

For the solutions A and B, the same chelating agent may be used, or different chelating agents may be used.

As a method in which the solution B is brought into contact with our support on which the nucleic acid is adsorbed, to elute the nucleic acid at the step c, the same method as the method of bringing the solution A into contact with the support and removing the solution A at the step b can be used. When at the step b, the solution A is passed through the column that houses our support, by passing the solution B after the solution A is passed, bringing the solution B into contact with the support and separating a liquid in which the nucleic acid is eluted from the support are performed by one operation, which is preferable. In this example, elution can be enhanced by standing or heating during contact of the solution B with the support. The time in standing is preferably 2 hours or less. The temperature in heating is preferably 70° C. or lower, and more preferably 50° C. or lower. When the liquid in which the nucleic acid is eluted is separated from the support, the liquid passing time is preferably 10 minutes or less.

When the solution in which the nucleic acid is eluted is separated from a mixture obtained by bringing the solution B into contact with the support on which the nucleic acid is adsorbed and the nucleic acid is collected at the step c, the same method as the method of removing the solution A at the step b can be used.

The collected nucleic acid can be subjected to chemical modification, if necessary. Examples of the chemical modification include modification of an end of the nucleic acid with a fluorescent dye, modification with a quencher, biotin modification, amination, carboxylation, maleimidization, succinimidization, phosphorylation, and dephosphorylation, and other examples thereof include dyeing by an intercalator. These modifications may be introduced by a chemical reaction, or may be introduced by an enzyme reaction. The amount of the nucleic acid can be determined indirectly by introducing modification groups before quantitative determination described above, and determining the amount of the modification groups introduced by the chemical modification instead of determining the amount of the collected nucleic acid. In the quantitative determination, the amount can be determined with high sensitivity since, the nucleic acid is collected, and in particular, a short-chain nucleic acid is collected at a high yield.

Our support is prepared by adsorbing a water-soluble neutral polymer on a surface of aluminum oxide. The surface coverage ratio of the polymer is preferably 7% or more, more preferably 10% or more, further preferably 20% or more, particularly preferably 30% or more, and the most preferably 40% or more. The water-soluble neutral polymer may not be necessarily adsorbed in an even thickness.

The coverage ratio of the polymer on alumina in our support is calculated by analyzing a potential map obtained by a surface potential microscope (also called Kelvin Force Microscope; KFM). As the surface potential microscope, for example, NanoScope Iva AFM Dimension 3100 Stage AFM System manufactured by Bruker AXS GmbH can be used.

The surface coverage ratio is calculated by the surface potential microscope at a scale in the field of view for measurement of 0.5 μm×1 μm. In a method of calculating the surface coverage ratio, a surface potential image of aluminum oxide is first obtained, and the average potential in the field of view is determined. Subsequently, the surface potential image of the water-soluble neutral polymer is obtained, and the average potential in the field of view is determined. The surface potential image of the aluminum oxide on which the water-soluble neutral polymer is adsorbed is then obtained, and the average potential in the field of view is determined. The coverage ratio of the aluminum oxide alone is considered as 0%, and the coverage ratio of the water-soluble neutral polymer alone is considered as 100%. The ratio of the average potential of the aluminum oxide on which the water-soluble neutral polymer is adsorbed to that of the water-soluble neutral polymer is obtained. Thus, the surface coverage ratio of the aluminum oxide on which the water-soluble neutral polymer is adsorbed is calculated. To calculate the surface coverage ratio, three particles of our support are randomly selected, and the average of measured values of the selected particles is used as the average potential in the field of view to be used.

Photoshop manufactured by Adobe Inc., can be used as an image analysis software when the surface coverage ratio is calculated. In this example, for image analysis, the average value of the surface potential of the aluminum oxide is set as a lower limit of the scale and the average value of the surface potential of the water-soluble neutral polymer is set as an upper limit of the scale. The lower limit color is set to black (8 bits, RGB value: 0), and the upper limit color is set to red (R value: 255), green (G value: 255), or blue (B value: 255) or the like. The surface potential image of the aluminum oxide on which the water-soluble neutral polymer is adsorbed is displayed at the set scales, the R, G, or B value is divided by 255, and the obtained ratio is considered as the surface coverage ratio.

Before the water-soluble neutral polymer is adsorbed on the surface, the aluminum oxide may be cleaned with a solution such as water or ethanol in advance, to remove an impurity that is adsorbed on the surface, or this washing operation may be omitted.

Examples of a method of adsorbing the water-soluble neutral polymer on the surface of the aluminum oxide include a method in which the water-soluble neutral polymer is dissolved to prepare a water-soluble neutral polymer solution, and the solution is brought into the contact with the aluminum oxide. Specifically, the aluminum oxide may be immersed in the water-soluble neutral polymer solution, the water-soluble neutral polymer solution may be added dropwise to the aluminum oxide, the water-soluble neutral polymer solution may be applied to the aluminum oxide, or the water-soluble neutral polymer solution may be sprayed onto the aluminum oxide in a mist form.

A method of immersing the aluminum oxide in the water-soluble neutral polymer solution is not particularly limited. For example, the solution may be stirred by pipetting or mixing by inversion, or with a disperser such as a stirrer, a mixer, a vortex, or a mill, a supersonic treatment instrument or the like.

The concentration of the water-soluble neutral polymer is not particularly limited, and is preferably 0.01 wt % or more, and more preferably 0.1 wt % or more.

The mixing time for stirring is not particularly limited as long as the water-soluble neutral polymer is evenly mixed with the aluminum oxide. When using a vortex, stirring is performed for 1 minute or more, and preferably 5 minutes or more.

The aluminum oxide can also be dip-coated with the water-soluble neutral polymer using a sifter, a sieve, or the like. When the polymer concentration is 0.1 wt % or more, the mixing time for immersion in the solution may be 5 minutes or more, and preferably 30 minutes or more.

When the water-soluble neutral polymer solution is added dropwise, a dropper, a dropping funnel, or the like can be used. When the polymer solution is added dropwise, the aluminum oxide may also be shaken or rotated, or a spin coater or the like may be used.

When the water-soluble neutral polymer solution is applied, a brush, a roller, or a wire bar can be used.

When the water-soluble neutral polymer solution is sprayed in a mist form, an air spray, an air brush, or the like can be used.

After the water-soluble neutral polymer is adsorbed on the aluminum oxide by the methods described above, a centrifugation operation may be performed to remove a supernatant polymer solution, or the aluminum oxide may be used for collection of a nucleic acid as it is without the centrifugation operation. When the polymer solution is dissolved in a solvent, the aluminum oxide may be used for collection of a nucleic acid with or without drying after the water-soluble neutral polymer is adsorbed on the aluminum oxide and the solvent is removed.

As our obtained support, a support that is prepared and stored may be used, or a support prepared at the time of use may be used.

When the obtained water-soluble neutral polymer is a solid, the water-soluble neutral polymer solution can be prepared by dissolving the water-soluble neutral polymer in water or an organic solvent. When the water-soluble neutral polymer is a solution, the water-soluble neutral polymer solution can be prepared by dilution. When the polymer is hard to dissolve or is hard to be mixed due to a high viscosity of the solution, a heating treatment or a supersonic treatment may be performed. As the organic solvent, for example, an organic solvent compatible with water such as ethanol, acetonitrile, methanol, propanol, tert-butanol, N,N-dimethylformamide (DMF), dimethylsulfoxide (DMSO), acetone, ethylene glycol, or glycerol is preferably used. When the polymer is hard to dissolve in water, the organic solvent described above may be added.

A support produced by covalently bonding the aluminum oxide and the water-soluble neutral polymer through a linker molecule or the like does not correspond to our support. Specific examples of the linker molecule include a silane coupling agent. A support produced by performing functionalization using such a silane coupling agent, forming an amide bond, an ester bond, a Michael addition reactant of thiol and maleimide, a disulfide bond, a triazole ring or the like, and fixing the polymer or the like does not correspond to our support.

Our kit for collection of a nucleic acid can be used to collect a nucleic acid from the sample containing a nucleic acid at a low elution volume compared to conventional methods. Our kit for collection of a nucleic acid contains as components our support of aluminum oxide having a surface where the water-soluble neutral polymer is adsorbed, the solution A containing 1 mM or more and 40 mM or less of a chelating agent, and the solution B containing 50 mM or more of a chelating agent. In addition to the components, the kit may contain a washing solution for washing the support on which the nucleic acid is adsorbed, an instruction such as a protocol for the method of collecting a nucleic acid and the like. The support, the solution A, and the solution B are housed in different containers before a collection treatment, and are taken from the respective containers at each step.

The support of aluminum oxide having a surface where the water-soluble neutral polymer is adsorbed, contained in the kit for collection of a nucleic acid may be in a dried state or a state in which the support is immersed in the water-soluble neutral polymer solution, or be housed in a column.

As the sample containing a nucleic acid, any solution containing a nucleic acid can be used. Examples of the nucleic acid include RNA, DNA, RNA/DNA (chimera), and an artificial nucleic acid. Examples of DNA include cDNA, microDNA (miDNA), genome DNA, synthetic DNA, cell-free DNA (cfDNA), ctDNA, and mitochondrial DNA (mtDNA). Examples of RNA include total RNA, mRNA, rRNA, miRNA, siRNA, snoRNA, snRNA, or non-coding RNA, precursors thereof, and synthetic RNA. Synthetic DNA and synthetic RNA can be artificially produced based on a predetermined base sequence (that may be a native sequence or a non-natural sequence), for example, using an automated nucleic acid synthesizer.

The sample containing a nucleic acid may be subjected to the following treatment, if necessary. This is because the nucleic acid in a biological sample is often encapsuled in a compound such as a cell membrane, a cell wall, a vesicle, a liposome, a micelle, a ribosome, a histone, a nuclear membrane, a mitochondrion, a virus capsid, an envelope, an endosome, or an exosome and they often interact with each other. To collect the nucleic acid at a higher yield, a treatment for releasing the nucleic acid from the compound may be performed.

Specifically, when a sample containing Escherichia coli is used, the following treatment may be performed to increase the collection efficiency of the nucleic acid. To a solution containing Escherichia coli, for example, a mixed solution of 0.2 M of sodium hydroxide and 1% SDS may be added (alkaline denaturation method), or a 10% sarkosyl solution may be added (non-denaturation method using sarkosyl). To the solution, lysozyme may be added. The sample may also be treated with proteinase K at 37° C. for 1 hour. As another method, a supersonic treatment may also be performed.

When a sample containing a yeast is used, the following treatment may be performed to increase the collection efficiency of the nucleic acid. For example, after a treatment with zymolyase commercially available from SEIKAGAKU CORPORATION or NACALAI TESQUE, INC., 10% SDS may be added.

When a sample containing a cell is used, the following treatment may be performed to increase the collection efficiency of the nucleic acid. For example, 1% SDS may be added. Another method may be addition of guanidium chloride, guanidine thiocyanate, urea or the like in a final concentration of 4 M or more. To this solution, sarkosyl may be added in a concentration of 0.5% or more. Mercaptoethanol may also be added in a concentration of 50 mM or more.

In the aforementioned operation, an inhibitor for a nuclease may be added to suppress decomposition of the nucleic acid. As an inhibitor of DNase, EDTA may be added in a concentration of 1 mM or less. “RNasin Plus Ribonuclease Inhibitor” (Promega Corporation), “Ribonuclease Inhibitor” (TAKARA BIO INC.), “RNase inhibitor” (TOYOBO CO., LTD.) or the like that is commercially available as an inhibitor for RNase can be used.

When the sample containing a nucleic acid contains DNA and RNA, DNA and RNA can be separated by phenol-chloroform extraction. For example, RNA and DNA are separated into an aqueous phase and a chloroform phase, respectively, by phenol-chloroform extraction under an acidic condition. RNA and DNA are dispersed into an aqueous phase by phenol-chloroform extraction under a neutral condition. The condition can be selected using such characteristics depending on the kind of the nucleic acid to be obtained. The chloroform may be replaced by p-bromoanisole.

In the phenol-chloroform extraction, “ISOGEN” (registered trademark: NIPPON GENE CO., LTD.), “TRIzol” (registered trademark: Life Technologies Japan Ltd.), RNAiso (Takara Bio Inc.), or “3D-GENE (registered trademark) RNA extraction reagent from liquid sample kit” (Toray Industries, Inc.), which are commercially available reagents, can be used. In the aforementioned treatments, one of the processes may be performed alone, or processes of different operations can be combined. The concentration of the solution used for the treatments can be changed, if necessary.

As the sample containing a nucleic acid, a solution in which a nucleic acid, an artificial nucleic acid, or a nucleic acid modified with a dye, phosphate group, or the like is dissolved, a liquid sample such as a body fluid, or a diluted solution thereof, or a diluted solution of a solid sample such as a cell pellet or a tissue piece may be used. When the sample containing a nucleic acid includes the solid sample, a solution obtained by any of the treatments for the sample may be used as it is as the sample containing a nucleic acid, or if necessary, the sample may be diluted and used. Even when the sample containing a nucleic acid is the liquid sample such as a body fluid, a solution obtained by any of the treatments for the sample may be used as it is as the sample containing a nucleic acid, or if necessary, the sample may be diluted and used, similarly to the solid sample. A solution for dilution is not particularly limited, and water or a solution generally used for dilution of a nucleic acid such as a Tris-hydrochloric acid buffer solution is preferably used. As a chaotropic salt, guanidium chloride, guanidine thiocyanate, or urea may be added in a final concentration of 4 M or more.

Adsorbing a nucleic acid on a support means adsorption in which reversible detachment is possible.

The collection rate of the nucleic acid adsorbed on the support can be determined as follows. The amount of the nucleic acid in the sample containing a nucleic acid is first calculated. Subsequently, an eluent is added to the support on which the nucleic acid is adsorbed, the amount of the nucleic acid in the solution after elution is calculated, and the amount of the nucleic acid eluted is calculated. The obtained value is used as the collection amount of the nucleic acid. The value is divided by the amount of the nucleic acid in the sample containing a nucleic acid to determine the collection rate of the nucleic acid.

Examples of a method of determining the amount of a nucleic acid include absorbance measurement, fluorescence measurement, luminescence measurement, electrophoresis, PCR, RT-PCR, analysis using a microarray, and analysis using a sequencer. The amount of an unmodified nucleic acid can be determined by absorbance measurement at 260 nm. In a nucleic acid modified with a fluorescent dye, the fluorescence intensity derived from the fluorescent dye is compared with the fluorescence intensity of a solution of a known concentration. Thus, the amount of the nucleic acid can be determined. In addition, quantitative determination can be performed by electrophoresis. In a method of calculating the collection rate by electrophoresis, a sample of a known concentration and a sample after a collection operation are simultaneously electrophoresed, a gel is dyed, and band concentrations are compared with each other by image analysis. Thus, the collection rate can be determined.

When the amount of the nucleic acid is too small to be determined, the yield of the nucleic acid can be compared by using a method of detecting a nucleic acid such as DNA chip or real-time PCR, and comparing detected values. In a measurement system in principle based on fluorescence measurement or luminescence measurement, for example, in a reaction of detection with DNA chip, the higher signal value can be interpreted as a higher yield. For example, in the DNA chip, a fluorescence image is obtained with a scanner, and the fluorescence signal intensity of each gene is converted into numbers. Thus, the yield can be compared. In a global analysis of expression amount of miRNA or mRNA, the fluorescence signal intensity of each gene can be compared. In a comparison of different procedures, the higher signal value can be interpreted as a higher yield. In an analysis of a plurality of kinds of genes, the sum of fluorescence signal (fluorescence signal total value) of each gene is obtained. In comparison of different procedures, the higher signal value can be interpreted as a higher yield. In rear-time PCR, an amplification curve is obtained by plotting the number of cycles on a horizontal axis and fluorescence intensities on a vertical axis. In this amplification curve, the numbers of cycles (Cq value and Ct value) when the signal intensities reach a certain signal intensity are each determined. In this example, the lower Ct value or Cq value can be interpreted as a higher yield. In cfDNA or genome DNA, a primer for a gene to be measured is designed. In a comparison of different collecting methods for the same primer, the lower Ct value or Cq value can be interpreted as a higher yield. For RNA such as miRNA or mRNA, measurement and detection can be achieved in the same manner as that for DNA except that a reverse transfer process is added. In this example, the lower Ct value or Cq value can be interpreted as a higher yield.

A polymer is a generic name for compounds in which a large number of repeating units, which are base units and are called monomer, are connected. A polymer used for our support includes a homopolymer consisting of one kind of monomer and a copolymer consisting of two or more kinds of monomers. The polymer also includes a polymer having any degree of polymerization. Furthermore, the polymer includes a natural polymer and a synthetic polymer.

The water-soluble neutral polymer used for our support is a polymer that is soluble in water, and has a solubility in water of at least 0.0001 wt % or more, preferably 0.001 wt % or more, more preferably 0.01 wt % or more, and further preferably 0.1 wt % or more.

The water-soluble neutral polymer used for our support is preferably a polymer having a zeta potential of −10 mV or more and +10 mV or less, more preferably −8 mV or more and +8 mV or less, further preferably −6 mV or more and +6 mV or less, and particularly preferably −4.0 mV or more and +1.1 mV or less, in a solution with a pH of 7.

The zeta potential is one of values representing electrical properties of an interface of a colloid in the solution. When a charged colloid is dispersed in the solution, an electric double layer is formed on a surface of the colloid by counter ions with respect to surface charges of the colloid. The electric potential on the surface of the colloid is called surface potential. The electric double layer is formed by an electrostatic interaction between the surface charges of the colloid. Therefore, ions are more strongly fixed on the electric double layer on a side of the colloid. In the electric double layer, a layer in which counter ions are strongly fixed on the surface of the colloid by the electrostatic interaction is called a stern layer, and the potential of the stern layer is called a stern potential. When the colloid is moved in the solution, the stern layer also moves with the colloid. In this process, a boundary surface that moves with the colloid exists outside the stern layer as viewed from the side of the colloid due to the viscosity of the solution. This surface is called a slipping plane or a slip plane. The potential of this slipping plane is defined as a zeta potential when the potential at a point sufficiently far from the colloid is defined as a zero point. Thus, the zeta potential varies depending on the surface charges of the colloid, and the surface charges vary according to protonation or deprotonation that depends on pH. The value in the solution with a pH of 7 is used as a standard. The distance from the surface of the colloid to the slipping plane is generally smaller than the size of the colloid, and therefore the surface of the colloid can be approximately represented as the slipping plane. Also in the water-soluble neutral polymer, the surface potential of the colloid dispersed in the solution can be considered as zeta potential.

The zeta potential can be determined using an electrokinetic phenomenon such as electrophoresis, electroosmosis, back flow potential, or precipitation potential, and can be measured by a method such as a microscopic method of electrophoresis, an electrophoresis method using a rotating diffraction grating method, a laser Doppler electrophoresis method, a supersonic vibration potential method, or an electroacoustic method. These measurements can be performed using a zeta potential measurement device. The zeta potential measurement device is commercially available from Otsuka Electronics Co., Ltd., Malvern Instruments Ltd., Ranku Brother Ltd., PenKem Inc., or the like.

The zeta potential can be measured using any of the devices. A laser Doppler electrophoresis method is general. The laser Doppler electrophoresis method is a measurement method using the Doppler effect in which the frequency of light or sound wave is changed when the light or sound wave hits an object in motion due to electrophoresis, and then scatters or reflects.

When the zeta potential of a polymer is measured, a polymer solution is prepared as a colloidal dispersion. Thus, the zeta potential can be measured. For example, the polymer is dissolved in an electrolyte such as a phosphate buffer solution, a sodium chloride solution, or a citrate buffer solution to prepare the polymer solution, scattered light and reflected light of the polymer dispersed in the solution is detected, and the zeta potential is measured. As the size of the colloid is larger, scattered light and reflected light can be detected at a lower concentration.

A specific condition for measurement of zeta potential of the polymer by the laser Doppler method is not particularly limited. For example, the polymer is dissolved in a phosphate buffer solution (10 mM, pH: 7) so that the concentration of the polymer is 1 wt % or more and 10 wt % or less, this solution is placed in a cell for measurement, and the cell is installed in a zeta potential measurement device in principle based on the laser Doppler electrophoresis method. Thus, the zeta potential can be measured at room temperature. For example, as the zeta potential measurement device, ELS-Z manufactured by Otsuka Electronics Co., Ltd., or the like can be used.

Examples of the water-soluble neutral polymer used for our support include the following. For example, a polyvinyl polymer such as polyvinyl alcohol or polyvinylpyrrolidone, a polyacrylamide polymer such as polyacrylamide, poly(N-isopropylacrylamide), or poly(N-(hydroxymethyl)acrylamide, a polyalkylene glycol polymer such as polyethylene glycol, polypropylene glycol, or polytetramethylene ether glycol, a cellulose such as poly(2-ethyl-2-oxazoline), (hydroxypropyl)methyl cellulose, methyl cellulose, ethyl cellulose, 2-hydroxyethyl cellulose, or hydroxypropyl cellulose, or the like can be used. A copolymer containing the aforementioned polymer can also be used.

Furthermore, the water-soluble neutral polymer used for our support also includes a polysaccharide or a polysaccharide analog such as ficoll, agarose, chitin, or dextran, a protein such as albumin, or a peptide.

A portion of a functional group of the water-soluble neutral polymer may be ionized or substituted with a functional group exhibiting positivity or negativity, or a functional group expressing water solubility such as an acetyl group may be introduced into a side chain.

For example, the molecular weight of the water-soluble neutral polymer preferably is preferably 0.4 kD or more, and more preferably 6 kD or more. The upper limit of the molecular weight is preferably 500 kD or less, and more preferably 150 kD or less. The molecular weight of the water-soluble neutral polymer is preferably within a range of 0.4 kD or more and 500 kD or less, and more preferably 6 kD or more and 150 kD or less.

Aluminum oxide used for our support is an amphoteric oxide represented by a composition formula of Al₂O₃, and is also called alumina.

For aluminum oxide, naturally generated aluminum oxide may be used, or industrially produced aluminum oxide may be used. Examples of a method of producing aluminum oxide include a Bayer process using gibbsite as a starting material, an alkoxide process through a hydroxide in a boehmite form (also called sol-gel process), a neutralization process, an oil droplet process, a thermal decomposition process of an aluminum salt, and an anode oxidation process.

Industrially produced aluminum oxide is available from reagent manufacturers, catalyst chemical manufacturers, the Committee of Reference Catalyst of the Catalysis Society of Japan, or the like.

Aluminum oxide is classified into α-aluminum oxide, ρ-aluminum oxide, χ-aluminum oxide, κ-aluminum oxide, η-aluminum oxide, γ-aluminum oxide, δ-aluminum oxide, and θ-aluminum oxide, depending on a crystal structure thereof. γ-aluminum oxide having a high specific surface area is preferred.

Acidic sites (Al⁺, Al—OH₂⁺) and basic sites (Al—O⁻) of aluminum oxide vary depending on the baking temperature during production. Depending on the number of acidic sites and basic sites of aluminum oxide, aluminum oxide is classified. When the number of acidic sites is large, aluminum oxide is acidic alumina. When the number of basic sites is large, aluminum oxide is basic alumina. When the number of acidic sites is substantially equal to the number of basic sites, aluminum oxide is neutral alumina. A difference in these properties can be confirmed by addition of a BTB solution that is a pH indicator. When aluminum oxide turns yellow by addition of a BTB solution, the aluminum oxide can be confirmed to be acidic alumina. When aluminum oxide turns green, the aluminum oxide can be confirmed to be neutral alumina. When aluminum oxide turns blue, the aluminum oxide can be confirmed to be basic alumina. Any aluminum oxide can be used regardless of such a difference in properties.

It is preferable that aluminum oxide be granular. The particle diameters may be the same, or particles having different particle diameters may be mixed and used. For example, aluminum oxide having a particle diameter of less than 212 μm can be preferably used. Aluminum oxide having a particle diameter of less than 100 μm can be more preferably used.

The particle diameter is defined by an aperture dimension of a sieve based on JIS Z-8801-1:2006 according to Japanese Industrial Standards. For example, a particle that passes through a sieve having an aperture of 40 μm and does not pass through a sieve having an aperture of 32 μm in accordance with the JIS standard has a particle diameter of 32 μm or more and less than 40 μm.

### EXAMPLES

Our methods and kits will be further specifically described using the following Examples. This disclosure is not interpreted to be limited to Examples.

### Material and Method

Polyethylene glycol was obtained from Merck Ltd. γ-aluminum oxide (N613N) was obtained from JGC Catalysts and Chemicals Ltd. Sodium polyphosphate (CAS No. 68915-31-1) was obtained from FUJIFILM Wako Pure Chemical Corporation.

Other reagents were obtained from Wako Pure Chemical Industries, Ltd., Tokyo Chemical Industry Co., Ltd., and Sigma-Aldrich Japan, and were used as they were particularly without purification. As a nucleic acid for measurement of collection rate, a nucleic acid synthesized by converting a nucleic acid having a base length of 22 of SEQ ID NO: 1 that was known as a let7a sequence of miRNA into a DNA sequence of SEQ ID NO: 2, and Cy3-labeling a 5′-terminal of the DNA sequence was obtained from Eurofins Genomics K.K. This nucleic acid was referred to as Cy3-DNA. The nucleic acid was used as it was particularly without purification.

As a mixer, “CUTE MIXER CM-1000” manufactured by TOKYO RIKAKIKAI CO., LTD., was used. As a centrifuge, CT15RE manufactured by Hitachi, Lid., was used.

From a healthy subject with informed consent, a human serum was collected using venoject II vacuum blood collection tube VP-AS109K60 (manufactured by Terumo Corporation).

A support according to our methodology was prepared as follows, and used in Examples and Comparative Examples. Each 20 mg of basic γ-aluminum oxide was weighed into a 1.5-mL tube. As an aqueous polymer solution, 200 μL of polyethylene glycol (PEG, 10 kD), which was a water-soluble neutral polymer, was added to the tube in a concentration of 10 wt %, and stirred for 10 minutes with a mixer.

Our support prepared above was housed in “Nanosep MF Centrifugal Devices (0.45 μm), to prepare a spin column housing the support. The spin column was used in Examples and Comparative Examples.

A solution B was prepared as follows. A 250 mM phosphate buffer solution (pH: 7) was first prepared. The concentration of polyphosphoric acid (250 mM) was determined by the molecular weight of phosphoric acid that was a structural unit, and the pH was adjusted to 7 by hydrochloric acid or sodium hydroxide. Equivalent amounts of 250 mM phosphoric acid and 250 mM polyphosphoric acid prepared above were mixed to obtain the solution B (125 mM phosphoric acid-125 mM polyphosphoric acid mixed solution (pH: 7)), and the solution B was used in Examples and Comparative Examples.

### Examples 1 to 11

As listed in Table 1, as a chelating agent for a solution A, 25 mM citric acid (pH: 7) (Example 1), 10 mM citric acid (pH: 7) (Example 2), 5 mM citric acid (pH: 7) (Example 3), 1 mM citric acid (pH: 7) (Example 4), 25 mM EDTA (pH: 7) (Example 5), 10 mM EDTA (pH: 7) (Example 6), 5 mM EDTA (pH: 7) (Example 7), 1 mM EDTA (pH: 7) (Example 8), 10 mM phosphoric acid (pH: 7) (Example 9), 5 mM phosphoric acid (pH: 7) (Example 10), and 1 mM phosphoric acid (pH: 7) (Example 11) were each used.

TABLE 1

| | Step b: Solution A | | Step c: | Adsorption | Elution | Collection |
|---|---|---|---|---|---|---|
| | Chelating agent | Concentration | Solution B | ratio [%] | ratio [%] | rate [%] |
| Example 1 | Citric acid | 25 mM | 125 mM | 85 | 81 | 69 |
| Example 2 | (pH: 7) | 10 mM | phosphoric | 89 | 80 | 71 |
| Example 3 | | 5 mM | acid-125 mM | 89 | 80 | 71 |
| Example 4 | | 1 mM | polyphosphoric | 91 | 58 | 53 |
| Example 5 | EDTA | 25 mM | acid mixed | 88 | 78 | 69 |
| Example 6 | (pH: 7) | 10 mM | solution | 88 | 78 | 69 |
| Example 7 | | 5 mM | (pH: 7) | 83 | 81 | 67 |
| Example 8 | | 1 mM | | 80 | 78 | 62 |
| Example 9 | Phosphoric | 10 mM | | 87 | 81 | 70 |
| Example 10 | acid | 5 mM | | 90 | 80 | 72 |
| Example 11 | (pH: 7) | 1 mM | | 88 | 79 | 70 |
| Comparative | None | | | 82 | 53 | 43 |
| Example 1 | | | | | | |

### Comparative Example 1

In Comparative Example 1, a nucleic acid was collected by the same operation as those in Examples 1 to 11 except for the step b under the same condition as those in Examples 1 to 11. Comparative Example 1 corresponds to the method of collecting a nucleic acid described in Patent Literature 1. The results are listed in Table 1.

Step a: As a sample containing a nucleic acid, a human serum containing 100 pmol of Cy3-DNA was used. 400 μL of 7 M GTN and 25 mM HEPES solution (pH: 7) in which 100 pmol of Cy3-DNA was dissolved, and 200 μL of human serum were mixed by pipetting, and the mixture was used as the sample containing a nucleic acid. To the spin column housing our support, the prepared sample containing a nucleic acid was added, and centrifuged (100 G, 10 minutes). A flow-through was then discarded, and the tube was then exchanged with a new collection tube.

Washing step 1 (first washing step): 350 μL of 50 mM HEPES buffer solution (pH: 7) was added to the spin column, followed by centrifugation (1,000 G, 2 minutes). A flow-through was then discarded, and the tube was then exchanged with a new collection tube.

Step b: 350 μL of each of 11 kinds of the solutions A was added to the spin column, followed by centrifugation (1,000 G, 2 minutes). A flow-through was then discarded, and the tube was then exchanged with a new collection tube.

Step c: 50 μL of the solution B was added to the spin column, and allowed to stand for 15 minutes. Subsequently, centrifugation (1,000 G, 2 minutes) was performed, and a flow-through was collected as a nucleic acid solution.

The adsorption ratio of the nucleic acid on the support was calculated by fluorescence measurement of Cy3 as follows. Before addition to the spin column housing the support, the fluorescence intensity of the sample containing a nucleic acid was first measured. The fluorescence intensity of the nucleic acid solution obtained at the step c was measured. A ratio of a value obtained by dividing the fluorescence intensity after passing through the spin column by the fluorescence intensity before passing through the spin column to 100 pmol that was the amount of the nucleic acid contained in the sample containing a nucleic acid was calculated, and the amount of the nucleic acid in the nucleic acid solution obtained at the step c was calculated. A difference between this value and 100 pmol that was the amount of the nucleic acid before passing through the column was obtained, and the amount of the adsorbed nucleic acid was calculated. The amount of the adsorbed nucleic acid was divided by 100 pmol that was the amount of the nucleic acid before addition of aluminum oxide, to calculate the adsorption ratio.

The elution ratio of the nucleic acid was calculated by fluorescence measurement of Cy3 as follows. 50 μL of the solution B was added to the support on which the nucleic acid was adsorbed, 150 μL of water was added to an eluent after elution, and fluorescence measurement was performed. Subsequently, 50 μL of the solution B in which 100 pmol of Cy3-DNA was dissolved was then prepared, 150 μL of water was added, and fluorescence measurement was performed. The fluorescence intensity of the eluent was divided by the fluorescence intensity of this solution, to calculate the amount of the eluted nucleic acid. The amount of the eluted nucleic acid was divided by the amount of the adsorbed nucleic acid, to calculate the elution ratio.

The collection rate of the nucleic acid was calculated by multiplying the obtained adsorption ratio by the elution ratio. The results of Examples 1 to 11 are listed in Table 1. As seen from the results, when at the step b, a step of bringing the solution containing 1 mM or more and 40 mM or less of a chelating agent into contact with the support on which the nucleic acid is adsorbed and removing the solution A is performed, the nucleic acid can be collected at a high yield compared to Comparative Example 1 not using the solution A.

### Example 12

At a collecting step of Example 1, the following washing step 2 (second washing step) was added between the steps b and c, and a nucleic acid was collected.

Washing step 2 (second washing step): 350 μL of 50 mM HEPES buffer solution (pH: 7) was added to the spin column, followed by centrifugation (1,000 G, 2 minutes). A flow-through was then discarded, and the tube was then exchanged with a new collection tube.

Other operation and condition were the same those in Example 1, to calculate the adsorption ratio, elution ratio, and collection rate of the nucleic acid. The results are listed in Table 2.

TABLE 2

| | Step b: Solution A | | | | | | |
|---|---|---|---|---|---|---|---|
| | Chelating | Concen- | Washing | Step c: | Adsorption | Elution | Collection |
| | agent | tration | step 2 | Solution B | ratio [%] | ratio [%] | rate [%] |
| Example 1 | Citric acid | 25 mM | None | 125 mM | 85 | 81 | 69 |
| | (pH: 7) | | | phosphoric acid | | | |
| | | | | −125 mM | | | |
| Example 12 | | 25 mM | 50 mM | polyphosphoric | 84 | 71 | 60 |
| | | | HEPES | acid mixed | | | |
| | | | buffer | solution (pH: 7) | | | |
| | | | solution | | | | |
| | | | (pH: 7) | | | | |

As seen from the results, even when the washing step 2 is added between the steps b and c, the nucleic acid can be collected at a high yield compared to Comparative Example 1 not using the solution A.

As seen from the results of Examples 1 to 12 and Comparative Example 1, the methods of Examples 1 to 12 that perform the step b enable collection of the nucleic acid at a high yield compared to the method of Comparative Example 1, that is, the method of Patent Literature 1.

### Examples 13 to 21

The nucleic acid was collected, and the adsorption ratio, elution ratio, and collection rate of the nucleic acid were calculated by the same operation as those in Examples 1 to 12 under the same condition as those in Examples 1 to 12 except that as a chelating agent for a solution A, 5 mM HEDP (pH: 7) (Example 13), 5 mM GMP (pH: 7) (Example 14), 10 mM NTMP (pH: 7) (Example 15), 5 mM NTMP (pH: 7) (Example 16), 1 mM NTMP (pH: 7) (Example 17), 5 mM EDTMP (pH: 7) (Example 18), 10 mM polyphosphoric acid (pH: 7) (Example 19), 5 mM polyphosphoric acid (pH: 7) (Example 20), and 1 mM polyphosphoric acid (pH: 7) (Example 21) were each used. The results are listed in Table 3.

TABLE 3

| | Step b: Solution A | | Step c: | Adsorption | Elution | Collection |
|---|---|---|---|---|---|---|
| | Chelating agent | Concentration | Solution B | ratio [%] | ratio [%] | rate [%] |
| Example 13 | HEDP | 5 mM | 125 mM | 94 | 73 | 68 |
| | (pH: 7) | | phosphoric acid | | | |
| Example 14 | GMP | 5 mM | −125 mM | 96 | 63 | 60 |
| | (pH: 7) | | polyphosphoric | | | |
| Example 15 | NTMP | 10 mM | acid mixed | 96 | 57 | 55 |
| Example 16 | (pH: 7) | 5 mM | solution | 93 | 78 | 72 |
| Example 17 | | 1 mM | (pH: 7) | 94 | 66 | 62 |
| Example 18 | EDTMP | 5 mM | | 93 | 70 | 65 |
| | (pH: 7) | | | | | |
| Example 19 | Polyphos- | 10 mM | | 96 | 66 | 63 |
| Example 20 | phoric | 5 mM | | 96 | 78 | 75 |
| Example 21 | acid | 1 mM | | 90 | 72 | 65 |
| | (pH: 7) | | | | | |

The results indicate that even when a phosphonic acid-based chelating agent is used as a chelating agent, the nucleic acid can be collected at a high yield.

### INDUSTRIAL APPLICABILITY

Our method of collecting a nucleic acid that enables collection of a trace amount of nucleic acid present in a body fluid at a high yield is industrially very applicable in collection of the nucleic acid from a sample containing a nucleic acid at a high yield.

1-9. (canceled)

10. A method of collecting a nucleic acid from a sample containing a nucleic acid using a support of aluminum oxide having a surface where a water-soluble neutral polymer is adsorbed, the method comprising steps a to c:
step a: a step of bringing the support into contact with the sample containing a nucleic acid to adsorb the nucleic acid on the support;
step b: a step of bringing the support on which the nucleic acid is adsorbed into contact with a solution A containing 1 mM or more and 40 mM or less of a chelating agent; and
step c: after the step b, a step of bringing the support on which the nucleic acid is adsorbed into contact with a solution B containing 50 mM or more of a chelating agent to elute the nucleic acid.

11. The method according to claim 10, wherein the chelating agent is a carboxylic acid-based chelating agent, a phosphoric acid-based chelating agent, or a phosphonic acid-based chelating agent.

12. The method according to claim 11, wherein the carboxylic acid-based chelating agent is citric acid, nitrilotriacetic acid, ethylenediaminetetraacetic acid, glycol ether diaminetetraacetic acid, and/or a salt thereof.

13. The method according to claim 11, wherein the phosphoric acid-based chelating agent is phosphoric acid, polyphosphoric acid, metaphosphoric acid, and/or a salt thereof.

14. The method according to claim 11, wherein the phosphonic acid-based chelating agent is 1-hydroxyethane-1,1-diphosphonic acid, glycine-N,N-bis(methylenephosphonic acid), nitrilotris(methylenephosphonic acid), 2-phosphonobutane-1,2,4-tricarboxylic acid, ethylenediamine tetramethylenephosphonic acid, and/or a salt thereof.

15. The method according to claim 10, wherein the support is housed in a column.

16. The method according to claim 10, wherein the water-soluble neutral polymer is a polymer having a zeta potential of −10 mV or more and +10 mV or less in a solution with a pH of 7.

17. The method according to claim 10, wherein the water-soluble neutral polymer is polyethylene glycol, polyvinyl alcohol, polyvinylpyrrolidone, poly(2-ethyl-2-oxazoline), or (hydroxypropyl)methylcellulose.

18. A kit for collection of a nucleic acid, the kit comprising a support of aluminum oxide having a surface where a water-soluble neutral polymer is adsorbed, a solution A containing 1 mM or more and 40 mM or less of a chelating agent, and a solution B containing 50 mM or more of a chelating agent.


SEQUENCE LISTING (2 sequences)

SEQ ID NO: 1 - RNA, 19 nt, Artificial Sequence
  Description of Artificial Sequence Synthetic oligonucleotide
aauggauuuuuggagcagg

SEQ ID NO: 2 - DNA, 22 bp, Artificial Sequence
  Description of Artificial Sequence Synthetic oligonucleotide
tgaggtagtaggttgtatagtt